(19) United States
(12) Patent Application Publication
(10) Pub. No.: US 2019/0282588 A1
(43) Pub. Date: Sep. 19, 2019

(54) POLYMERIC EXTENDED RELEASE COMPOSITIONS OF HYDROXYPROGESTERONE CAPROATE AND METHODS OF USING SAME

(71) Applicants: AMAG Pharmaceuticals, Inc., Waltham, MA (US); Evonik Corporation, Parsippany, NJ (US)

(72) Inventors: Tom Tice, Indian Springs, AL (US); Gary Winchester, Warrior, AL (US); Kevin Burton, Hoover, AL (US); Michael J. Jozwiakowski, Sudbury, MA (US); Robert Birch, Saint Charles, MO (US); Kyle Haraldsen, Woburn, MA (US); Jeff Caplette, Fiskdale, MA (US)

(21) Appl. No.: 16/301,237

(22) PCT Filed: May 16, 2017
(86) PCT No.: PCT/US2017/032816

§ 371 Date: Nov. 13, 2018

Related U.S. Application Data
(60) Provisional application No. 62/336,869, filed on May 16, 2016.

(51) Int. Cl. A61K 31/57; A61K 9/16

(52) U.S. Cl. CPC A61K 31/57; A61K 9/1647

(57) ABSTRACT

The disclosure is directed in part to extended release compositions that include hydroxy progesterone caproate. For example, provided herein is a therapeutic microparticle composition comprising a plurality of microparticles, wherein the microparticles each comprise poly (lactide-co-glycolide) and hydroxyprogesterone caproate.

### CROSS-REFERENCE TO RELATED APPLICATION

This application claims the benefit of and priority to U.S. Provisional Patent Application Ser. No. 62/336,869, filed May 16, 2016, the contents of which are hereby incorporated by reference.

### BACKGROUND

Preterm delivery is a major health problem in the United States and worldwide. Preterm delivery is often defined as delivery before 37 completed weeks of gestation and has been reported to be the major determinant of infant mortality in developed countries. Preterm delivery is more common in the United States than in many other developed countries, and is predominantly responsible for the relatively high rate of infant mortality in the United States as compared to many other developed countries. Over the past two decades, the rate of preterm delivery in the United States has been reported to have increased from 9% to 11%. In addition to preterm delivery, various other pregnancy-related conditions are major health problems in the United States and worldwide. These include, for example, the delivery of low birth weight neonates, delivery of small for gestational age neonates, pregnancy-related complications, fetal mortality, neonatal morbidity, neonatal mortality, infant morbidity, infant mortality, and childhood developmental delays.

Preterm delivery and other pregnancy-related conditions such as the delivery of low birth weight neonates and/or small for gestational age neonates have serious health, societal, and economic costs. For example, preterm delivery and the delivery of low birth weight neonates and/or small for gestational age neonates can lead to neonatal morbidity, longer stays in the neonatal intensive care unit, and a higher risk of long term morbidities including, for example, cerebral palsy, mental retardation, and learning disabilities.

A number of risk factors for preterm delivery and other pregnancy-related conditions (e.g., previous pregnancy resulting in preterm delivery, previous delivery of low birth weight and/or small for gestational age neonates) have been identified. For example, women who have had a previous spontaneous preterm delivery are at high risk for preterm delivery in subsequent pregnancies. Other risk factors for preterm delivery include: tobacco use during pregnancy (e.g., smoking); infection; multiple gestations (twins, triplets, etc.); alcohol use, abuse, or dependence during pregnancy; substance use, abuse, or dependence during pregnancy; poor nutrition during pregnancy; stress, anxiety, and/or depression; insufficient weight gain during pregnancy; advanced maternal age; African-American descent; and low socio-economic status. Tobacco use or exposure, in particular smoking, during pregnancy is a significant risk factor for preterm delivery and other undesirable maternal, fetal, and neonatal outcomes.

Weekly injection of steroids such as 17-alpha-hydroxyprogesterone caproate (“17-HPC” or “HPC”) have been used to reduce the risk of preterm birth, but such injections can be painful, and patient compliance can be difficult, especially since the injection usually must be administered by a health professional. There is a need for an alternative route of administration that may, for example, significantly reduce the number of injections and increase the likelihood of patient compliance.

### SUMMARY

Provided herein, in an embodiment, is a therapeutic microparticle composition comprising a plurality of microparticles, wherein the microparticles each comprise: about 25 to about 50 weight percent poly (lactide-co-glycolide) having an inherent viscosity of about 0.16 dL/g to about 0.28 dL/g, wherein the inherent viscosity is measured at 25° C., at a concentration of 0.1% w/v in chloroform; and about 50 to about 75 weight percent hydroxyprogesterone caproate, wherein the therapeutic microparticles having a mean particle size of about 30 μm to about 95 μm. Microparticles disclosed herein, in an embodiment, have a substantially core-shell morphology, where e.g., the shell at least partially encompasses the core, for example, where the shell is substantially poly(lactide-co-glycolide) and the core is substantially hydroxyprogesterone caproate. For example, the hydroxyprogesterone caproate may be substantially crystalline.

For example, disclosed microparticles may include about 1 to about 20 weight, or about 4 to about 12 weight percent, based on the total weight of the hydroxyprogesterone caproate, of crystalline hydroxyprogesterone caproate characterized by a powder X-ray diffraction pattern having characteristic peaks in degrees 2θ at about 9.6, about 12.2, and about 18.3.

For example, provided herein is a therapeutic microparticle comprising: about 25 to about 50 weight percent biocompatible, bioabsorbable polymer; and crystalline hydroxyprogesterone caproate, wherein at least a portion of the crystalline hydroxyprogesterone caproate is Form B, characterized by a powder X-ray diffraction pattern having characteristic peaks in degrees 2θ at about 9.6, about 12.2, and about 18.3, wherein the biocompatible, bioabsorbable polymer is for example, poly(lactic acid), poly (lactide-co-glycolide), or a mixture thereof, and/or the crystalline hydroxyprogesterone caproate comprises about 1 to about 20 weight percent based on the total weight of the hydroxyprogesterone caproate, Form B.

Provided herein, in an embodiment, is a unit dose comprising a disclosed therapeutic microparticle composition or therapeutic microparticles wherein the unit dose has about 750 to about 1000 mg of the hydroxyprogesterone caproate. For example provided herein is a unit dose vial or pre-loaded syringe for delivering about 750 mg to about 1000 mg hydroxyprogesterone caproate comprising a disclosed therapeutic microparticle composition or disclosed therapeutic microparticles.

Also provided herein is a kit comprising: a first container comprising a disclosed therapeutic microparticle composition or disclosed therapeutic microparticles; and a second container comprising a pharmaceutically acceptable diluent (e.g., phosphate buffered saline solution) for the therapeutic microparticle composition. A dual chamber cartridge is also provided, for example, in which one of the chambers comprises a disclosed therapeutic microparticle composition or disclosed therapeutic microparticles and the other chamber optionally comprises a diluent.

In another embodiment, a method of reducing the risk of preterm birth in a pregnant human patient in need thereof, comprising administering a disclosed pharmaceutically acceptable microparticle composition.

### BRIEF DESCRIPTION OF THE FIGURES

FIG. 1 shows the images of a disclosed microparticle (optical image (1A) and raman spectroscopy image (1B)) showing core-shell morphology of the microparticle.

FIG. 2 shows the images of a disclosed microparticle (optical image (2A) and raman spectroscopy image (2B)) showing core-shell morphology of the microparticle.

FIG. 3 shows the image of a disclosed microparticle (optical image (3A) and raman spectroscopy image (3B)) showing core-shell morphology of the microparticle.

FIG. 4 shows the image of a disclosed microparticle (optical image (4A) and raman spectroscopy image (4B)) showing core-shell morphology of the microparticle.

FIG. 5 shows the optical image of a disclosed microparticle (optical image (5A) and raman spectroscopy image (5B)) showing core-shell morphology of the microparticle.

FIG. 6 shows the plasma concentration (ng/mL) of hydroxyprogesterone caproate of disclosed microparticle compositions in a rat model.

### DETAILED DESCRIPTION

The disclosure is generally directed at least in part to therapeutic microparticle compositions comprising a plurality of microparticles, wherein the microparticles each comprise a bioabsorbable polymer (e.g., a biocompatible and/or substantially biodegradable polymer) and 17-alpha-hydroxyprogesterone caproate (HPC, 17-HPC). For example, provided herein are microparticles having hydroxyprogesterone caproate and a polymer selected from poly(lactide) (e.g., poly(L-lactide) and/or poly(DL-lactide)), polyglycolide, poly(ester amide), poly(lactide-co-glycolide “PLG”) (e.g., poly(L-lactide-co-glycolide) (L-PLG) or poly(DL-lactide-co-glycolide) (DL-PLG), or mixtures thereof.

In general, representative examples of bioabsorbable polymers that may be used in embodiments of the present disclosure include, but are not limited to, poly(N-acetylglucosamine) (chitin), chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-glycolide); poly(caprolactone), poly(DL-lactide-co-caprolactone), poly(L-lactide-co-caprolactone), poly(trimethylene carbonate), poly(ethylene amide), polyethylene acrylate, poly(glycolic acid-co-trimethylene carbonate), co-poly(ether-esters) (e.g., PEO/PL), poly(ester amides), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Bioabsorbable polymers that may be useful in various embodiments of the disclosure include polydioxanone (PDO), polyhydroxyalkanoate, polyhydroxybutyrate, poly(glycerol sebacate), or copolymers or derivatives including these and/or other polymers. Bioabsorbable polymers generally refer to polymers that are capable of being completely resorbed without degradation and/or degraded and/or eroded when exposed to bodily fluids such as blood and can be gradually resorbed, absorbed, and/or eliminated by the body. The processes of breaking down and absorption of the polymer can be caused by, for example, hydrolysis and metabolic processes.

Disclosed microparticles, in certain embodiments, may includepoly(lactide-co-glycolide (PLG). The term “microparticles” as used herein contemplates microcapsules, and/or nanoparticles. PLG is a biocompatible and bioabsorbable copolymer of lactide and glycolide synthesized by the ring opening of lactide and glycolide monomers. PLGs can made to have various ratios of lactide and glycolide in their copolymer backbones PLGs and PLs can be linear or branched homopolymers and copolymers, e.g., depending on the initiator used (for example, e.g., lauryl alcohol may be used to prepare linear polymers; glucose may be used to prepare branched polymers (star polymers). Other initiators, including polyethylenes (PEGs), are also contemplated. The lactide monomer can be L-lactide, D-lactide or DL-lactide. PLG to be used in accordance with the present disclosure can be characterized, for example, by a lactide:glycolide mole ratios of about 45 to about 100, or about 50-75 lactide to about 75 to about 100, or about 85:15 lactide:glycolide, about 75:25 lactide:glycolide, about 65:35 lactide:glycolide, about 60:40 lactide:glycolide, about 50:50 lactide:glycolide, about 40:60 lactide:glycolide, about 25:75 lactide:glycolide, or about 15:85 lactide:glycolide. For example, a disclosed microparticle may include poly(lactide-co-glycolide) with a lactide:glycolide mole ratio of about 45-75 lactide to about 55-25 glycolide, e.g., about 65 to about 35 glycolide. It is appreciated that certain contemplated polymers, e.g., poly(lactide-co-glycolide) (PLG) may have an acid end group (e.g., —COOH), or a ester or other end group that may be one of PEG, lauryl, ethyl, methyl or other end group.

Disclosed microparticles, in certain embodiments, may contain poly(lactic acid-co-glycolic acid) (PLGA). PLGA is a biocompatible and bioabsorbable copolymer of lactic acid and glycolic acid synthesized by polycondensation of lactic acid and glycolic acid. PLGAs can made to have various ratios of lactic acid and glycolic acid in their copolymer backbones. PLGAs can be linear or branched homopolymers and copolymers. A lactic acid monomer that forms a PLGA (or PLA) can be L-lactic acid, D-lactic acid or DL-lactic acid. PLGA to be used in accordance with the present disclosure can be characterized, for example, by a lactic acid:glycolic acid mole ratio of about 45 to about 100, or about 50-75 lactide to about 75 to about 100, or about 85:15 lactic acid:glycolic acid, about 75:25 lactic acid:glycolic acid, about 65:35 lactic acid:glycolic acid, about 60:40 lactic acid:glycolic acid, about 50:50 lactic acid:glycolic acid, about 40:60 lactic acid:glycolic acid, about 25:75 lactic acid:glycolic acid, or about 15:85 lactic acid:glycolic acid. For example, a disclosed microparticle may include poly(lactic acid-co-glycolic acid) with a lactic acid:glycolic acid mole ratio of about 45-75 lactic acid to about 55-25 glycolic acid, e.g., about 65 to about 35 glycolic acid. It is appreciated that certain contemplated polymers, e.g., poly(lactic acid-co-glycolic acid) will have an acid end group, e.g., —COOH.

In certain embodiments, a disclosed microparticle includes a bioabsorbable polymer such as poly (lactide-co-glycolide) having an inherent viscosity of about 0.1 dL/g to about 1.0 dL/g, e.g., about 0.16 dL/g to about 0.28 dL/g, or about 0.35 to about 0.45 dL/g, or about 0.16 to about 0.5 dL/g, where the inherent viscosity is measured at 25° C., at a concentration of 0.1% w/v in chloroform, with a size 0b Ubbelohde glass capillary viscometer. For example, a disclosed microparticle includes a bioabsorbable polymer such as poly (lactide-co-glycolide) having a number average molecular weight of about 15 to about 25 kDa.

For example, provided herein are microparticles having about 10 to about 90 weight percent, or about 20 to about 90, or about 20 to about 80, about 20 to about 60, or about 25 to about 50 weight percent poly (lactide-co-glycolide), and about 50 to about 75 weight percent 17-alpha-hydroxyprogesterone caproate (i.e. hydroxyprogesterone caproate (“HPC)) or about 10 to about 90 weight percent, about 20 to about 90 weight percent, about 40 to about 80 weight percent, about 45 to about 75 weight percent, about 50 to about 60 weight percent, or about 55 to about 65 weight percent hydroxyprogesterone caproate (e.g., about 45, 50, 55, 57, 60, 62, 65, 67, 70 weight percent hydroxyprogesterone caproate). Such disclosed therapeutic microparticles may have a mean particle size of about 30 μm to about 95 μm, or about 30 μm to about 60 μm, about 30 μm to about 50 μm, about 35 μm to about 55 μm, or about 30 μm to about 50 μm, before or after sterilization, e.g., achieved through beam sterilization or gamma radiation. For example, disclosed microparticles may have a D10 diameter (where 10% of a sample has smaller particles) of about 30 μm to about 50 μm, or and/or have a D90 diameter of about 50 μm to about 70 μm, or about 50 μm to about 60 μm. Particle mean size or mean size distribution may be measured by laser diffraction.

Disclosed microparticles, in some embodiments, have a substantially core-shell morphology, wherein the polymer is substantially in the shell domain and the HPC is substantially in the core domain. For example, a disclosed core-shell microparticle shell may at least partially encompasse the core. For example, a disclosed therapeutic microparticle may include a shell having substantially poly(lactide-co-glycolide) and the core is substantially hydroxyprogesterone caproate. Disclosed microparticles having a core-shell morphology may have a shell with a thickness of about 3 μm to about 10 μm, or about 4 μm to about 9 μm. For example, such disclosed microparticles with core-shell morphology may also have a high loading of HPC, e.g. at least about 40 weight percent HPC, or at least about 45 weight percent or more HPC.

In an embodiment, a disclosed microparticle composition has total non-aqueous solvent levels below about 3.0 weight percent, or below about 2.0 weight percent.

Microparticles, as disclosed herein, may include substantially crystalline hydroxyprogesterone caproate. HPC may be present in a disclosed microparticle in one or more polymorphic crystalline forms. For example, HPC may be present in Form A and/or Form B.

Crystalline Form A HPC is characterized by a powder X-ray diffraction pattern having at least one or more characteristic peaks in degrees 2θ at about 7.3, 14.1, and 15.4, for example, crystalline form A can be characterized by a powder X-ray diffraction pattern having at least one or more characteristic peaks in degrees 2θ at about 7.3, 12.5, 14.0, 14.1, 15.4, 16.4, and/or 19.7; e.g., at about 7.3, 9.8, 12.5, 14.0, 14.1, 15.4, 16.4, 16.9, 17.7, and 19.7; or at about 7.3, 9.8, 12.5, 13.0, 13.5, 14.0, 14.1, 15.4, 16.4, 16.9, 17.7, 19.2, 19.7, and 24.2. Form A HPC may be characterized by a differential scanning calorimetry profile with an endothermic peak from about 120° C. to about 124° C.

Crystalline Form B HPC is characterized by a powder X-ray diffraction pattern having at least one or more characteristic peaks in degrees 2θ at about 9.6, 12.2, and 18.3, for example, having at least one or more characteristic peaks in degrees 2θ at about 9.6, 12.2, 13.9, 14.8, 15.4, 18.3, and 19.2, or at about 3.9, 9.6, 12.2, 13.0, 13.9, 14.8, 15.4, 18.3, 19.2, and 30.7.

The term “about” in the context of peaks at degrees 2θ means that there is an uncertainty in the measurements of the 2θ of ±0.5 (expressed in 2θ) or that there is an uncertainty in the measurements of the 2θ of ±0.2 (expressed in 2θ). The powder X-ray diffraction pattern of the crystalline forms were obtained using Cu Kα radiation

Provided herein are therapeutic microparticle compositions, in an embodiment, wherein the microparticles comprises about 1 to about 20 weight percent (e.g., about 4 to about 12 weight percent or about 5 to about 10 weight percent), based on the total weight of the hydroxyprogesterone caproate, of crystalline hydroxyprogesterone caproate characterized by a powder X-ray diffraction pattern having characteristic peaks in degrees 2θ at about 9.6, about 12.2, and about 18.3.

For example, provided herein, in an embodiment, is a therapeutic microparticle comprising: a biocompatible, bioabsorbable polymer such as polymer described herein (e.g., poly(lactic) acid, poly (lactide-co-glycolide), or a mixture thereof) (wherein, for example, the microparticle includes about 25 to about 60 weight percent polymer); and crystalline hydroxyprogesterone caproate, wherein at least a portion of the crystalline hydroxyprogesterone caproate is Form B, characterized by a powder X-ray diffraction pattern having characteristic peaks in degrees 2θ at about 9.6, about 12.2, and about 18.3. For example, a disclosed microparticle may include 1 to about 20 weight percent (or about 4 to about 12 weight percent) Form B HPC, based on the total weight of the hydroxyprogesterone caproate.

Also provided herein is a therapeutic microparticle composition comprising a disclosed therapeutic microparticle and a pharmaceutically acceptable diluent (for example a phosphate buffered solution, optionally further comprising carboxymethyl cellulose and/or polyoxyethylene (20) sorbitan monolaurate.

Upon parenteral administration of a disclosed microparticle composition to a patient, in certain embodiments, the patient maintains an effective plasma concentration of at least 2 ng/mL of the hydroxyprogesterone caproate at seven days, at fourteen days or even at twenty-one days after administration. In certain embodiments, upon parenteral administration of the composition to a patient, the patient maintains an effective plasma concentration of 4 ng/mL of the hydroxyprogesterone caproate at 14 days or more (e.g., at 21 days) after administration. Parenteral administration may be for example intramuscular, or subcutaneous administration.

### Methods of Treating

Described herein are methods for reducing the occurrence of preterm delivery and/or reducing the occurrence of other pregnancy-related conditions such as delivery of low birth weight neonates, delivery of small for gestational age neonates, pregnancy-related complications, fetal mortality, neonatal morbidity, neonatal mortality, infant morbidity, infant mortality, and childhood developmental delays in a human (or other mammalian, e.g., horse, cow, goat, ewe, cat, dog, rat or mouse) female patient (e.g., a human or mammalian patient pregnant with a singleton or with multiple fetuses), comprising administering a disclosed microparticle composition. For example, provided herein is a method of reducing the risk of preterm birth in a pregnant human patient (wherein one or more risks are described below for example, e.g., wherein the pregnant human patient (e.g., having a singleton pregnancy) has a history of singleton spontaneous preterm birth) in need thereof, comprising administering a pharmaceutically acceptable disclosed.

For example, methods disclosed herein are effective for reducing the occurrence of preterm delivery in a pregnant human subject at risk for preterm delivery. Risk factors for preterm delivery and/or other pregnancy-related conditions include previous preterm delivery, exposure to tobacco smoke, exposure to tobacco smoke residue, use of smokeless tobacco, substance use or abuse or dependence, alcohol use or abuse or dependence, stress, anxiety, depression, poor nutritional status, insufficient weight gain during pregnancy, advanced maternal age, low socio-economic status, and combinations thereof.

Disclosed methods can include, in some embodiments, administering (e.g., subcutaneously, intravenously, or intramuscularly administering) to a patient a disclosed pharmaceutically acceptable microparticle composition every two weeks, monthly, every two months, or every 6 months. For example, a pharmaceutically acceptable composition or microparticle composition may be administered starting at 16 weeks, 0 days of gestation or after in a human patient. Such disclosed pharmaceutically acceptable compositions or microparticle composition may be administered, in an embodiment, about monthly (or about every three weeks, or about every two weeks), and then if needed, a second composition comprising hydroxyprogesterone caproate is administered weekly, until week 37 of gestation or delivery, whichever occurs first. For example, disclosed methods may include administration of a disclosed composition wherein the patient maintains an effective plasma concentration of the hydroxyprogesterone caproate for at least two, three, four or five weeks upon administration of a single dose.

Contemplated methods as disclosed herein, when relating to subcutaneous administration of a disclosed composition, such administration may be into e.g., the upper anterior thigh, buttocks, upper arm (e.g., triceps area), or abdomen of the patient.

As noted, preterm delivery is a major health problem in the U.S. and worldwide. Preterm delivery is often defined to include any delivery before 37 weeks or before 35 weeks of gestation. The gestational age of an embryo or fetus may be calculated using ultrasound and/or from the date of the woman's last menstrual period or from 14 days before conception if the date of conception is known. For purposes of determining the effectiveness of the methods of the present invention, preterm delivery can be defined as any live birth occurring prior to 37 weeks of gestation, prior to 36 weeks of gestation, or prior to 35 weeks of gestation. Since viability may occur for live births prior to 35 weeks of gestation, preterm delivery may also be defined as any live birth occurring between 20 and 36 weeks of gestation.

Also contemplated herein in part are methods of reducing the risk of delivering a low-birth-weight infant in a pregnant human patient, comprising administering to the patient a disclosed composition. Neonates having a relatively low-birth-weight and/or relatively small size are generally associated with a higher risk of various complications as compared to neonates having a weight and/or size within normal ranges, including an increased risk for neonatal morbidity and mortality, and infant morbidity and mortality. As used herein, the term “low-birth-weight neonates” encompasses low-birth-weight neonates (neonates having a weight at birth of less than about 2500 g (about 5.5 pounds)), very low birth weight neonates (neonates having a weight at birth of less than about 1500 g (about 3.3 pounds)), and extremely low birth weight neonates (neonates having a weight at birth of less than about 1000 g (about 2.2 pounds)). A neonate is suitably classified as a small for gestational age neonate if his or her weight at birth is below the 10th percentile for gestational age, as measured according to the accepted standards published by Battaglia et al., or if birth weight and/or length are at least 2 standard deviations (SDs) below the mean for gestational age, as described by Lee et al. See Battaglia et al., A Practical Classification of Newborn Infants by Weight and Gestational Age, J. Pediatrics 71(2):159-63 (August 1967) and Lee et al., International Small for Gestational Age Advisory Board Consensus Development Conference Statement: Management of Short Children Born Small for Gestational Age, Apr. 24-Oct. 1, 2001, Pediatrics 111(6 Pt. 1):1253-61 (June 2003), both of which are incorporated by reference herein for all relevant purposes.

Contemplated herein in an embodiment are methods of reducing the risk pregnancy related complications in a pregnant human patient, comprising administering to the patient a disclosed composition. Pregnancy-related complications contemplated include, for example, placental abruption, placenta previa, and hypertension-related disorders (e.g., preeclampsia and eclampsia). These complications are generally known to contribute to preterm delivery, delivery of low birth weight neonates, etc. Thus, reducing the occurrence of these complications likewise reduces the occurrence of preterm delivery, delivery of low birth weight neonates, etc.

Also contemplated herein are methods of reducing the risk of neonatal mortality in a pregnant human patient, comprising administering to the patient a disclosed composition. Fetal mortality includes any death of a fetus at 20 weeks of gestation or later or any death of a fetus weighing more than 500 g. Fetal mortality includes both antepartum deaths (i.e., deaths occurring before birth) and intrapartum deaths (i.e., deaths occurring during labor and delivery). Neonatal mortality refers to the death of a live-born neonate within the first 28 days of life. Neonatal mortality includes both early neonatal mortality (i.e., death of a live-born neonate within the first seven days of life) and late neonatal mortality (i.e., death of a live-born neonate after the first seven days of life but within the first 28 days of life). Together, fetal mortality and early neonatal mortality are often referred to as “perinatal mortality.” Thus, “perinatal mortality” refers to deaths occurring between 20 weeks of gestation and the end of the 7th day after delivery. Infant mortality includes deaths which occur after 28 days of life, but before one year. Also contemplated herein are methods of reducing the risk of neonatal morbidity and/or development delays in a neonate comprising administering to the patient a disclosed composition Neonatal morbidity and infant morbidity refer to any disease, disorder, symptom, or other undesirable outcome occurring in a neonate or an infant, respectively. Developmental delays occur when children have not yet reached expected developmental milestones by the expected time period. Neonatal morbidity, infant morbidity, and childhood developmental delays encompass a number of conditions affecting neonates, infants, and/or children, including, but not limited to, transient tachypnea, respiratory distress syndrome, bronchopulmonary dysplasia, a need for ventilatory support/mechanical ventilation, a need for supplemental oxygen, intraventricular hemorrhage, necrotizing enterocolitis, patent ductus arteriosus, retinopathy, sepsis, sudden infant death syndrome (SIDS), cerebral palsy, mental retardation, learning disabilities, and behavioral disorders. Various additional diagnoses associated with neonatal morbidity, infant morbidity, and/or childhood developmental delays include anemia, arthritis, asthma, diabetes, diarrhea, colitis, ear infections, eczema, food or digestive allergies, hay fever, respiratory allergies, seizures, severe headaches or migraines, sickle cell disease, and stuttering and stammering. Other conditions include communication problems, problems with problem solving, attention or learning problems (e.g., attention-deficit hyperactivity disorder (ADHD)), autism, problems carrying out activities and problems with coordination.

An embodiment provided herein is a method of reducing the risk of preterm birth in a pregnant human patient (e.g., a human patient that has one or more risk factors (e.g., one or more previous preterm births and/or another risk factor as outlined below)), comprising administering to the patient a disclosed composition. A variety of risk factors that may be associated with the above-listed pregnancy-related conditions alone or in combination are detailed below. An exemplary risk factor is a patient that has a history of singleton spontaneous preterm birth. Various risk factors listed below are in connection with exposure to tobacco (e.g., tobacco smoke or tobacco smoke residue). Other risk factors that may contribute to and/or cause one or more pregnancy-related conditions include substance use or abuse or dependence, alcohol use or abuse or dependence, stress, poor nutritional status, insufficient weight gain during pregnancy, advanced maternal age, low socio-economic status, and combinations thereof. Behaviors unfavorable to a subject's health such as smoking tend to cluster (e.g., women who smoke are also more likely to have poor diets). Thus, many women exhibit more than one risk factor for the pregnancy-related conditions, which may increase the risk of occurrence of the pregnancy-related conditions. For example, the occurrence of more than one of the following risk factors are commonly exhibited by a single subject: exposure to tobacco smoke, stress, poor nutritional status, low socio-economic status, alcohol use, abuse, or dependence. Thus, in various preferred embodiments the methods of the present invention are directed to reducing the occurrence of one or more pregnancy-related conditions in a pregnant human subject exhibiting at least one risk factor selected from the group consisting of exposure to tobacco smoke, stress, poor nutritional status, low socio-economic status, alcohol use or abuse or dependence, and combinations thereof.

One significant risk factor for preterm delivery and the other pregnancy-related conditions is exposure of the pregnant human to tobacco smoke during pregnancy. This exposure may occur in many forms. Exposure to tobacco smoke includes smoking of tobacco products by the pregnant human subject herself, as well as passive smoking via the inhalation of smoke from tobacco products used by others (commonly referred to as second-hand smoke or environmental tobacco smoke). In either case, the tobacco smoke may be smoke generated by the use of, for example, a cigarette, a cigar, or a pipe, or any other implement which generates smoke from tobacco. A primary means of exposure of subjects to tobacco smoke in accordance with the present invention is smoking by the pregnant human subject.

Substance use, abuse, or dependence includes the use or abuse of, or the dependence on, drugs commonly referred to as “street drugs” (e.g., marijuana and cocaine) and/or the use or abuse of, or the dependence on, prescription drugs other than as directed by a physician. Alcohol use, abuse, or dependence generally includes the use or abuse of, or the dependence on, any alcohol-containing product, such as beer, wine, or liquor. Alcohol use may specifically refer to confirmed use of alcohol during pregnancy. High risk alcohol use during pregnancy is defined as confirmed use of alcohol sufficient to produce high blood alcohol levels (100 mg/dL or greater) delivered at least weekly in early pregnancy.

Experiencing relatively high stress levels may put pregnant women at an increased risk for one or more of the above-noted pregnancy-related conditions. Stress levels are suitably measured by a method well known to one skilled in the art, for example, by psychometric scales including the stress component of the Abbreviated Scale for the Assessment of Psychosocial Status in Pregnancy tool, the Stressful Life Events scale (part of the CDC's Pregnancy Risk Assessment and Monitoring System (PRAMS)) and the Modified Life Experiences Survey. A stress level exceeding the pre-defined values for one of these scales would generally be considered to increase the risk for the pregnancy-related conditions discussed above. Stress may be caused, for example, by life events such as divorce, illness, injury, job loss, or the like.

Poor nutritional status may put a pregnant human at an increased for the one or more of the above-noted pregnancy-related conditions. Nutritional status may be assessed by weight gain during pregnancy based on pre-pregnancy body mass index (BMI) according to the Institute of Medicine recommendations. See Institute of Medicine, Weight Gain During Pregnancy: Reexamining the Guidelines (2009), which is incorporated by reference herein for all relevant purposes. For example, a pregnant human subject will generally be considered to have a poor nutritional status if weight gain during pregnancy is insufficient according to these guidelines.

Generally, as maternal age increases so too does the risk of occurrence of preterm delivery and/or one or more other pregnancy-related conditions. By advanced maternal age, it is meant that the pregnant human subject is at least 35 years of age at the time of delivery. A pregnant human subject is suitably considered to have a low socio-economic status if the pregnant human subject's family and/or household income is at or below the federal poverty level.

Contemplated treatments of a pregnant human subject with a disclosed microparticle composition typically begins during the first or second trimester of pregnancy (i.e., during weeks 1-27 of gestation) and continues until relatively late in the third trimester or until delivery, whichever occurs first. However, it is anticipated that the benefits of the disclosed methods will still be realized even if the treatment is not initiated until the third trimester. Thus, for example, treatment with a disclosed microparticle composition is typically initiated at between 1 week and about 35 weeks of gestation and continues until about 37 weeks of gestation, or delivery, whichever occurs first. Alternatively, a disclosed treatment is suitably initiated at between about 12 weeks and about 30 weeks of gestation and continues until about 36 weeks of gestation, or delivery, whichever occurs first. In some methods of treatment, the treatment (e.g., administration of a disclosed microparticle composition) is initiated at between about 16 weeks, zero days, to about 21 weeks (or 20 weeks, six days of gestation, or in another embodiment, initiated at about 25 weeks) and continues until about 36 weeks or 37 weeks of gestation, or until delivery of an infant, whichever occurs first. In an embodiment, a disclosed method of treatment is initiated during the second or third trimester. Thus, in accordance with various disclosed methods of treatment, treatment with a disclosed microparticle composition is typically initiated at 13 weeks of gestation or later (e.g., at or around 28 weeks of gestation or later for a human patient).

### Kits and Unit Doses

Pharmaceutical compositions contemplated by this disclosure (e.g., compositions that include disclosed microparticles) may include about 700 milligrams (mg) to about 1400 mg of 17-HPC. For example, this disclosure contemplates a unit dose of a composition that includes disclosed microparticles and having about 750 to about 1250 mg HPC, or about 750 mg to about 1000 mg HPC.

Also contemplated herein is a unit dose vial or pre-loaded syringe for delivering about 750 mg to about 1000 mg (or about 750 to about 1250 mg) hydroxyprogesterone caproate to a patient comprising a disclosed therapeutic microparticle composition or disclosed therapeutic microparticles.

In another embodiment, this disclosure provides for a kit comprising: a first container comprising a disclosed therapeutic microparticle composition or a disclosed therapeutic microparticle; and a second container comprising a pharmaceutically acceptable diluent for the therapeutic microparticle composition. Such pharmaceutically acceptable diluent may be a phosphate buffered saline solution, that in certain embodiments, further comprises carboxymethyl cellulose and/or polyoxyethylene(20) sorbitan monolaurate.

A dual chamber cartridge, is also contemplated herein, in which one of the chambers comprises a disclosed therapeutic microparticle composition or a disclosed therapeutic microparticle and the other chamber optionally comprises a diluent.

### EXAMPLES

The examples which follow are intended in no way to limit the scope of this invention but are provided to illustrate aspects of the disclosed methods. Many other embodiments of this invention will be apparent to one skilled in the art.

### Example 1 Microparticle Preparation

A. Preparation of 2 wt % Poly(Vinyl Alcohol) (PVA) (CP=Continuous Phase)

Water is added to a vessel along with PVA and this mixture is heated to 92±2° C. during stirring. Once the temperature reaches 92±2° C., the resultant PVA solution is cooled to ambient temperature and filtered. Ethyl acetate is added to the PVA solution to bring the solution up to the desired final volume.

B. Preparation of a PLG/HPC Solution (DP=Dispersed Phase)

Ethyl acetate is charged into vessel followed by addition of 65:35 PLG with an inherent viscosity of about 0.2 dL/g. The mixture is stirred until dissolution of the 65:35 PLG is observed. Once the 65:35 PLG is in solution, HPC is added to the 65:35 PLG solution and this mixture is stirred for a minimum of 4 hours and until the dissolution of the HPC is observed.

C. HPC Microparticle Formation

The PVA solution (CP) and the PLG/HPC solution (DP) are passed together under laminar flow through a glass-bead packed-bed column. The column was packed with glass beads with a bead diameter range of 400-600 microns as described in U.S. Pat. No. 8,916,196. As micro-droplets emerge out of the glass-bead, packed-bed column, as an oil-in-water emulsion, the droplets encounter a stream of fresh water which extracts ethyl acetate, the PLG/HPC solvent, out of the microdroplets and hardens them into microparticles. The resultant suspension of hardened microparticles is then stirred for 60±5 minutes. The hardened microparticles are isolated on a vorti-sieve. Next the collected microparticles are added to fresh water and stirred at 4±2° C. for 2 hours followed by a second collection on a vorti-sieve. The microparticles are then dried under high vacuum with a nitrogen overlay to obtain a dry powder. The scale of the mentioned microparticle process ranged from 20 to 1000 grams.

Microparticles prepared according to above were prepared at a 1,000-g scale. Some of these batches were terminally sterilized with E-beam radiation (noted as ES in the tables) at 25 kGy. The residual solvent/residual volatiles for each batch were determined to be about 1.22 to about 1.36 wt %. Microencapsulated HPC purity ranged from 99.8 to 100%.

TABLE 1
Microparticles prepared according to this example

| | Polymer | Target | Cold | | |
|---|---|---|---|---|---|
| | Solution | HPC | Rinse | | HPC |
| Batch | Consentration, | Loading, | Time, | Batch | Content, |
| Number | wt % | wt %. | min | Yield, % | wt % |
| 899-034 | 15 | 60 | 130 | 55 | 57.3 |
| 899-105-1 | 15 | 60 | 120 | 54 | 57.3 |
| 899-105-1ES | 15 | 60 | 120 | 54 | 58.1 |
| 899-106-2 | 15 | 60 | 120 | 63 | 58.0 |
| 899-106-2ES | 15 | 60 | 120 | 63 | 58.1 |
| 899-113-3 | 12.5 | 65 | 120 | 63 | 63.8 |
| 899-113-3ES | 12.5 | 65 | 120 | 63 | 62.5 |
| 899-144-4 | 10 | 70 | — | 69 | 66.9 |
| 899-144-4ES | 10 | 70 | — | 69 | 69.9 |

TABLE 2
Particle size data for microparticle batches prepared according to this
example.

| Batch Number | Mean Size, μm | D₁₀, μm | D₉₀, μm |
|---|---|---|---|
| 899-034 | 45.0 | 31.3 | 65.7 |
| 899-105-1 | 45.4 | 33.4 | 59.7 |
| 899-105-1ES | 44.4 | 33.6 | 57.4 |
| 899-106-2 | 43.7 | 33.8 | 55.1 |
| 899-106-2ES | 43.2 | 33.6 | 54.1 |
| 899-113-3 | 47.4 | 33.7 | 67.6 |
| 899-113eS | 41.0 | 31.8 | 52.6 |
| 899-144-4 | 39.5 | 30.5 | 51.0 |
| 899-144-4ES | 42.1 | 32.1 | 52.8 |

### Example 2—X-Ray Diffraction Analysis

HPC microparticle samples were analyzed using a XRD method and then evaluated using multivariate technique. All the analyzed microparticles showed some polymorph Form A with some Form B. Table 2 shows the concentration of Form A and Form B and particle size.

The quantification analysis method is based on using the whole XRD pattern between 2θ=6 to 10.39° and after background removal. X-ray powder diffraction patterns were obtained using a Bruker D8 Advance X-Ray Diffractometer equipped with a Cu Kα radiation source (λ=1.54060° A) in locked/coupled mode. Samples were placed on zero-background, silicon plate holders. The step was 0.05°. Count times were 1.3 second per step. The collection were done between 2θ=4° to 21°. Sample holder rotation during acquisition is required.

Preparation of samples was as follows. Accurately weigh approximately about 20 mg of microparticle solid. Add the whole quantity to the zero-background, silicon plate holder and spread it with spatula. Apply slight pressure using spatula to make a flat surface. When the analysis is completed, remove the background using the XRD software and export the whole pattern as xy data. The data in the range of 2θ=6 to 10.39° are used for prediction.

The calibration matrix is prepared using samples of the pure forms A and B. The XRD response is a function of the XRD machine used for analysis. Therefore, when the XRD machine is changed, the XRD of the pure samples of Forms A and B should be collected again prior to analysis for estimations.

Table 3 indicates the proportion of Forms A and B in HPC microparticle.

| Batch Number | Polymorph A, wt % | Polymorph B, wt % |
|---|---|---|
| 899-034 | 95 | 5 |
| 899-105-1 | 91 | 9 |
| 899-105-1ES | 90 | 10 |
| 899-106-2 | 94 | 6 |
| 899-106-2ES | 94 | 6 |
| 899-113-3 | 89 | 11 |
| 899-113-3ES | 90 | 10 |
| 899-144-4 | 90 | 10 |
| 899-144-4ES | 91 | 9 |

### Example 3—Optical and Raman Analysis of Microparticles

Raman spectra/imaging x-y (lateral scans) was conducted using WITecConfocal Raman Microscope (model: Alpha 300R). X-y\Y scanning areas vary from 46 μm×46 μm to 50 μm×50 μm, and the pixels per image range from 92×92 pixels to 100×100 pixels, e.g., four pixels per square μm. The integration time per pixel is 0.20 second; Laser wavelength=532 nm; Spectrograph grating=600 g/mm; CCD detector=1024 pixels; CCD temperature=−60° C.

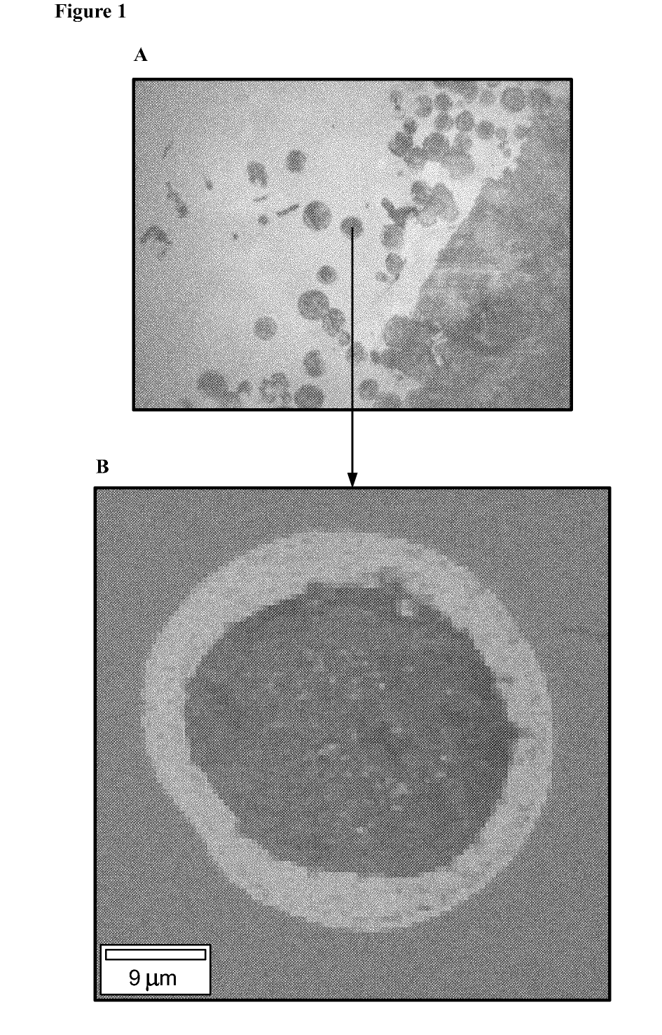

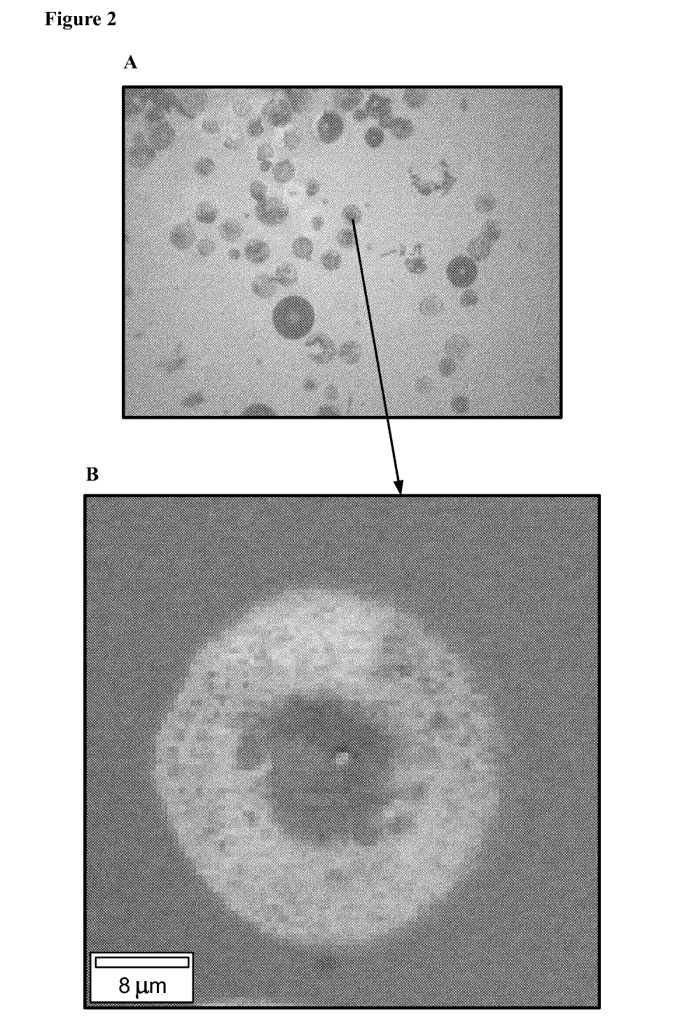

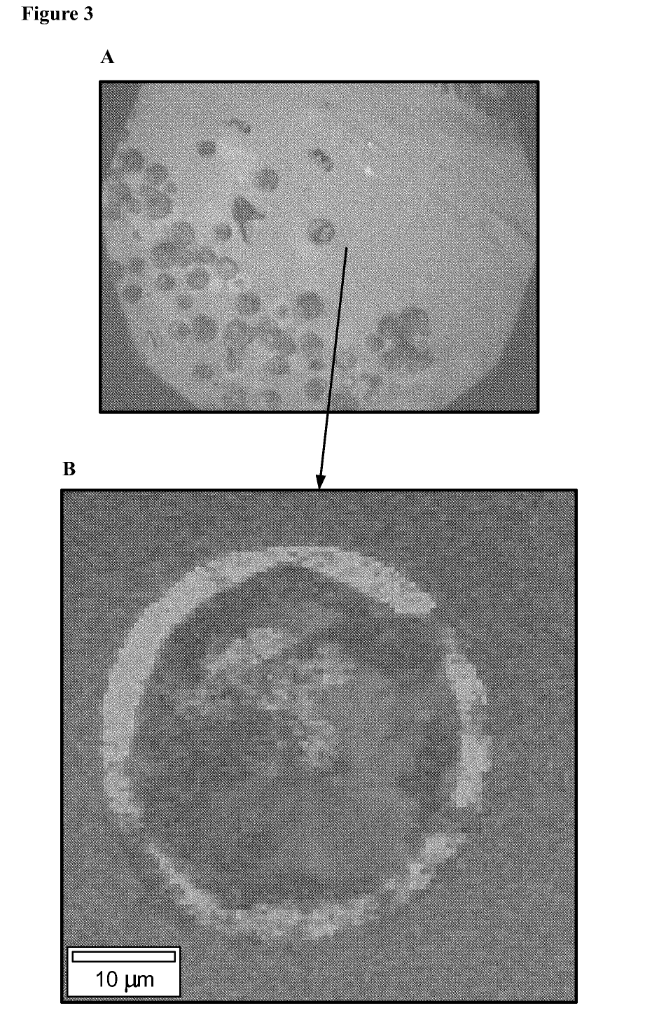

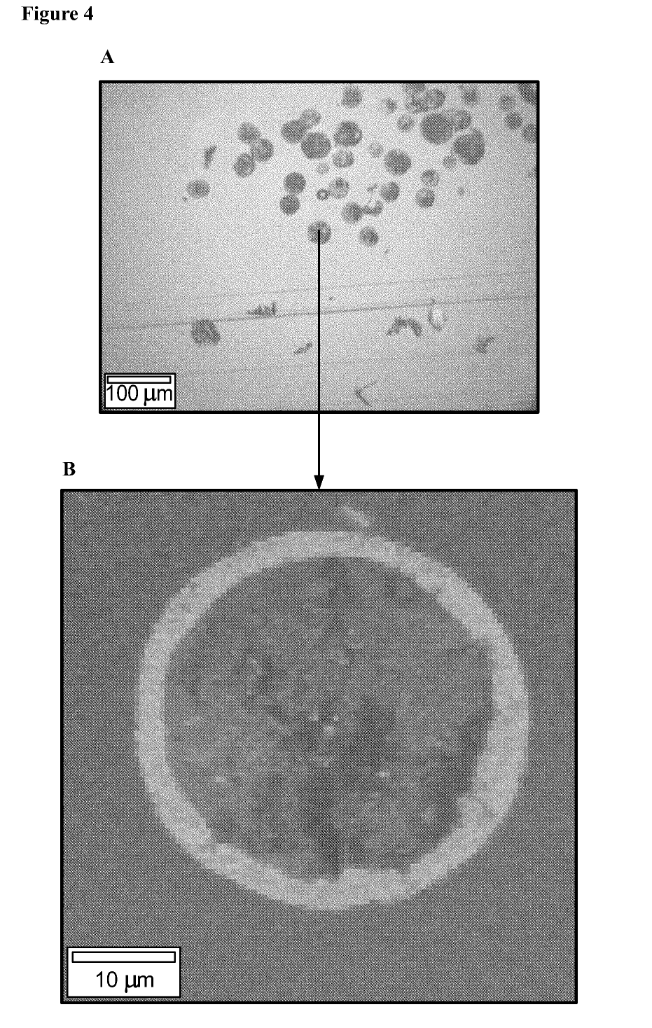

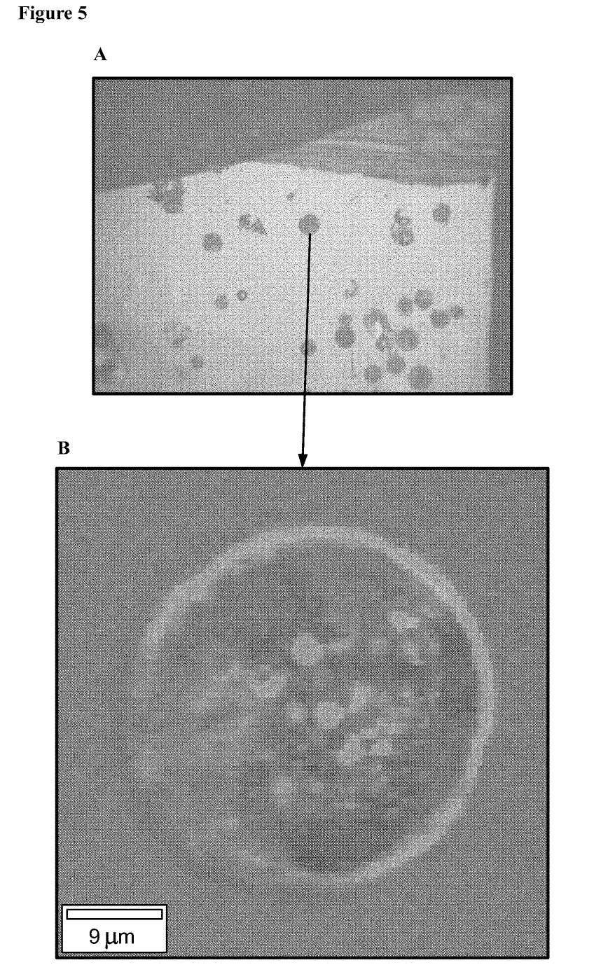

FIG. 1 shows the optical image of microparticle Batch 1 (optical image (A) and raman spectroscopy image (B), showing the core-shell morphology, where the outer ring is poly lactide-glycolide, and inner core is substantially HPC. FIG. 2 shows the optical image of microparticle Batch 2 (optical image (A) and raman spectroscopy image (B), showing the core-shell morphology, where the outer ring (shell) is poly(lactide-glycolide), and inner core is substantially HPC. FIG. 3 shows the optical image of microparticle Batch 3 (optical image (A) and raman spectroscopy image (B), showing the core-shell morphology, where the outer ring is poly lactide-glycolide, and inner core is substantially HPC. FIG. 4 shows the optical image of microparticle Batch 3ES (optical image (A) and raman spectroscopy image (B), showing the core-shell morphology, where the outer ring is poly lactide-glycolide, and inner core is substantially HPC. FIG. 5 shows the optical image of microparticle Batch 4ES (optical image (A) and raman spectroscopy image (B), showing the core-shell morphology, where the outer ring is (poly lactide-glycolide), and inner core is substantially HPC.

### Example 4 Pharmokinetic Study in Female Sprague-Dawley Rats

Plasma samples for determination of the pharmacokinetics of HPC-loaded microparticles in the female Sprague-Dawley rat following intramuscular (IM) injection of various test and reference (control/Makena®-hydroxyprogesterone caproate in castor oil/benzyl benzoate/benzyl alcohol) formulations are conducted. A total of 42 female Sprague-Dawley [Crl:CD®(SD)] rats (including extras) were initially assigned to study. Six animals were assigned to each group. The animals are not fasted prior to dosing. For each dose, the weight of the dosing syringe for each animal was recorded prior to dosing and following dosing. Unless otherwise indicated, intramuscular doses was administered via bolus injection into the large muscle mass in the left and/or right hind limb(s) of each animal. Each animal in Group 1 received a weekly intramuscular dose of the appropriate test article formulation on Days 1, 8, 15, and 22, as outlined in the study design table above.

Test article administration on Days 8, 15, and 22 take place within ±30 minutes of the time of test article administration on Day 1.

Each animal in Group 2 received a single intramuscular dose of the appropriate test article formulation as outlined in the study design table above. The total dose is split between two injection sites as outlined in the study design table above. Blood collection times for the animals in Group 2 is calculated from the time that the second injection is administered.

Each animal in Groups 3-6 received a single intramuscular dose of the appropriate test article formulation suspended in an aqueous carboxymethyl cellulose/Tween 20 injection vehicle as outlined in the table below.

TABLE 4
Dosing protocol for the animal study

| | | | Dosing | Target 17-α | |
|---|---|---|---|---|---|
| | | HPC content, | on | HPC | Target Dose |
| Group | Treatment | wt % | Day | Dose Level | Volume |
| 1 | Control | NA | 1, 8, 15, | 5 mg/kg/dose | 0.1 mL/animal |
| | | | 22 | (20 mg/kg | (~0.36 mL/kg/dose) |
| | | | | cumulative) | |
| 2 | Control | NA | 1 | 20 mg/kg/dose | 0.4 mL/animal, split |
| | | | | (20 mg/kg | between 2 sites |
| | | | | cumulative) | (~1.45 mL/kg/dose) |
| 3 | Depot 1 | 60% | 1 | 20 mg/kg/dose | 0.2 mL/animal |
| | (HPC | | | (20 mg/kg | (~0.73 mL/kg/dose) |
| | microparticles) | | | cumulative) | |
| 4 | Depot 2 | 60% | 1 | 20 mg/kg/dose | 0.2 mL/animal |
| | (HPC | | | (20 mg/kg | (~0.73 mL/kg/dose) |
| | microparticles) | | | cumulative) | |
| 5 | Depot 4 | 60% | 1 | 20 mg/kg/dose | 0.2 mL/animal |
| | (HPC | | | (20 mg/kg | (~0.73 mL/kg/dose) |
| | microparticles) | | | cumulative) | |
| 6 | Depot 3 | 70% | 1 | 20 mg/kg/dose | 0.2 mL/animal |
| | (HPC | | | (20 mg/kg | (~0.73 mL/kg/dose) |
| | microparticles) | | | cumulative) | |

TABLE 5
treatment protocol

| Group | Treatment | Pharmacokinetic Blood Collection Intervals |
|---|---|---|
| 1 | HPC/Castor | 2, 4, 8, 12 (Day 1), 24 (Day 2), 48 (Day 3), |
| | Oil Control^{A} | 172 (Day 8), 244 (Day 11), 340 (Day 15), |
| 2 | HPC/Castor Oil | 412 (Day 18), 508 (Day 22), 676 (Day 29), |
| | Control (4X)^{A} | 844 (Day 36), 1012 (Day 43), and |
| 3 | Depot 1 (HPC | 1348 (Day 57) hours postdose |
| | microparticles) | |
| 4 | Depot 2 (HPC | |
| | microparticles) | |
| 5 | Depot 4 (HPC | |
| | microparticles) | |
| 6 | Depot 3 (HPC | |
| | microparticles) | |

FIG. 6 indicates the HPC plasma levels v. hours after administration of the control and depot formulations (HPC microparticle formulations).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention an and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A therapeutic microparticle composition comprising a plurality of microparticles, wherein the microparticles each comprise:
about 25 to about 50 weight percent poly (lactide-co-glycolide) having an inherent viscosity of about 0.16 dL/g to about 0.28 dL/g, wherein the inherent viscosity is measured at 25° C., at a concentration of 0.1% w/v in chloroform; and
about 50 to about 75 weight percent hydroxyprogesterone caproate, wherein the therapeutic microparticles having a mean particle size of about 30 μm to about 95 μm.

2. The therapeutic microparticle composition, wherein the microparticles have a substantially core-shell morphology.

3. The therapeutic microparticle composition of claim 2, wherein the shell at least partially encompasses the core.

4. The therapeutic microparticle composition of claim 2 or 3, wherein the shell at least partially encompasses the core.

5. The therapeutic microparticle composition of any one of claims 2-4, where the shell is substantially poly(lactide-co-glycolide) and the core is substantially hydroxyprogesterone caproate.

6. The therapeutic microparticle composition of any one of claims 2-5, wherein the shell has a thickness of about 3 μm to about 10 μm.

7. The therapeutic microparticle composition of any one of claims 1-6, wherein the particle size distribution is measured by laser diffraction.

8. The therapeutic microparticle composition of any of claims 1-7, wherein the poly(lactide-co-glycolide) has a lactide:glycolide mole ratio of about 45-75 lactide to about 55-25 glycolide.

9. The therapeutic microparticle composition of any one of claims 1-8, wherein the poly(lactide-co-glycolide) has an acid end group.

10. The therapeutic microparticle composition of any one of claims 1-9, wherein the microparticle comprises about 55 to about 70 weight percent hydroxyprogesterone caproate.

11. The therapeutic microparticle composition of any one of claims 1-10, wherein the total non-aqueous solvent levels is below about 3.0 weight percent.

12. The therapeutic microparticle composition of any one of claims 1-11, wherein the hydroxyprogesterone caproate is substantially crystalline.

13. The therapeutic microparticle composition of any one of claims 1-12, wherein the microparticles comprises about 1 to about 20 weight percent, based on the total weight of the hydroxyprogesterone caproate, of crystalline hydroxyprogesterone caproate characterized by a powder X-ray diffraction pattern having characteristic peaks in degrees 2θ at about 9.6, about 12.2, and about 18.3.

14. The therapeutic microparticle composition of any one of claims 1-13, wherein the microparticles comprise about 4 to about 12 weight percent, based on the total weight of the hydroxyprogesterone caproate, of crystalline hydroxyprogesterone caproate characterized by a powder X-ray diffraction pattern having characteristic peaks in degrees 2θ at about 9.6, about 12.2, and about 18.3.

15. A therapeutic microparticle comprising:
about 25 to about 50 weight percent biocompatible, bioabsorbable polymer; and
crystalline hydroxyprogesterone caproate, wherein at least a portion of the crystalline hydroxyprogesterone caproate is Form B, characterized by a powder X-ray diffraction pattern having characteristic peaks in degrees 2θ at about 9.6, about 12.2, and about 18.3.

16. The therapeutic microparticle of claim 15, wherein the biocompatible, bioabsorbable polymer is poly(lactic acid), poly (lactide-co-glycolide), or a mixture thereof.

17. The therapeutic microparticle of claim 15 or 16, wherein the crystalline hydroxyprogesterone caproate comprises about 1 to about 20 weight percent based on the total weight of the hydroxyprogesterone caproate, Form B.

18. The therapeutic microparticle of any one of claims 15-17, wherein the crystalline hydroxyprogesterone caproate comprises about 4 to about 12 weight percent based on the total weight of the hydroxyprogesterone caproate, Form B.

19. A therapeutic microparticle composition comprising a therapeutic microparticle of any one of claims 15-18 and a pharmaceutically acceptable diluent.

20. The therapeutic microparticle composition of claim 19, wherein the diluent further comprising a phosphate buffered saline solution.

21. The therapeutic microparticle composition of claim 19 or 20, further comprising carboxymethyl cellulose and/or polyoxyethylene (20) sorbitan monolaurate.

22. The therapeutic microparticle composition of any one of claims 19-21, wherein upon parenteral administration of the composition to a patient, the patient maintains an effective plasma concentration of 2 ng/mL of the hydroxyprogesterone caproate at seven days or at fourteen days after administration.

23. The therapeutic microparticle composition of any one of claims 19-22, wherein upon parenteral administration of the composition to a patient, the patient maintains an effective plasma concentration of 4 ng/mL of the hydroxyprogesterone caproate at 14 days after administration.

24. The therapeutic microparticle composition of claim 22 or 23, wherein parenteral administration is intramuscular, or subcutaneous administration.

25. A unit dose comprising the therapeutic microparticle composition of any one of claims 1-14 or the therapeutic microparticles of any one of claims 15-18 wherein the unit dose has about 750 to about 1000 mg of the hydroxyprogesterone caproate.

26. A unit dose vial or pre-loaded syringe for delivering about 750 mg to about 1000 mg hydroxyprogesterone caproate comprising the therapeutic microparticle composition of any one of claims 1-14 or the therapeutic microparticles of any one of claims 15-18.

27. A kit comprising:
a first container comprising the therapeutic microparticle composition of any one of claims 1-14 or the therapeutic microparticles of any one of claims 15-18; and
a second container comprising a pharmaceutically acceptable diluent for the therapeutic microparticle composition.

28. The kit of claim 27, wherein the pharmaceutically acceptable diluent comprises phosphate buffered saline solution.

29. The kit of claim 28, wherein the pharmaceutically acceptable diluent further comprises carboxymethyl cellulose and/or polyoxyethylene (20) sorbitan monolaurate.

30. A dual chamber cartridge, in which one of the chambers comprises the therapeutic microparticle composition of any one of claims 1-14 or the therapeutic microparticles of any one of claims 15-18 and the other chamber optionally comprises a diluent.

31. A method of reducing the risk of preterm birth in a pregnant human patient in need thereof, comprising administering the pharmaceutically acceptable microparticle composition of any one of claims 19-21.

32. The method of claim 31, wherein the pregnant human patient has a history of singleton spontaneous preterm birth.

33. The method of any one of claim 31 or 32, wherein the pregnant human patient has a singleton pregnancy.

34. The method of any one of claims 31-33, wherein the pharmaceutically acceptable microparticle composition is administered every two weeks, monthly, every two months, or every 6 months.

35. The method of any one of claims 31-34, wherein the pharmaceutically acceptable composition or microparticle composition is administered starting at 16 weeks, 0 days of gestation or after.

36. The method of any one of claims 31-35, wherein the pharmaceutically acceptable composition or microparticle composition is administered monthly, and then if needed, a second composition comprising hydroxyprogesterone caproate is administered weekly, until week 37 of gestation or delivery, whichever occurs first.

37. The method of any one of claims 31-36, wherein the patient maintains an effective plasma concentration of the hydroxyprogesterone caproate for at least three or four weeks upon administration of a single dose.
